(19) United States
(12) Patent Application Publication
(10) Pub. No.: US 2006/0122705 A1
(43) Pub. Date: Jun. 8, 2006

(54) Hydroxyapatite backed glenoid prosthesis

(76) Inventor: Jeffrey D. Morgan, Florence, SC (US)

Correspondence Address: Sara A. Centioni;Nexsen Pruet, LLC, PO Box 10648, Greenville, SC, 29603-0648, US

(21) Appl. No.: 11/294,799

(22) Filed: Dec. 5, 2005

Related U.S. Application Data
(60) Provisional application No. 60/633,552, filed on Dec. 6, 2004.

(51) Int. Cl. A61F 2/40

(52) U.S. Cl. 623/19.11

(57) ABSTRACT

A glenoid prosthesis for use in total shoulder arthroplasty. The glenoid prosthesis has a concave articulating plate that can interact with the humeral component of an artificial shoulder joint, and a backing for connecting and bonding the prosthesis to the human scapula. The backing of the glenoid prosthesis can be made using hydroxyapatite, which forms a bond with human bone. The backing can further include means for connecting, such as pegs or a keel, to facilitate the connection of the glenoid prosthesis with the scapula.

### CROSS REFERENCE TO RELATED APPLICATIONS

The present application claims the benefit of priority of U.S. Application No. 60/633,552 filed on Dec. 6, 2004.

### STATEMENT REGARDING FEDERALLY SPONSORED RESEARCH OR DEVELOPMENT

Not Applicable.

### REFERENCE TO A SEQUENCE LISTING, A TABLE, OR A COMPUTER PROGRAM LISTING COMPACT DISK APPENDIX

Not Applicable.

### BACKGROUND OF THE INVENTION

The present invention relates generally to shoulder joint prostheses, and, more specifically, to the glenoid component of shoulder joint prostheses.

The replacement of the natural shoulder joint with an artificial one is referred to as total shoulder arthroplasty. An artificial shoulder joint typically includes a humeral component that is implanted within the humerus and a glenoid component that is connected and fixed to the scapula. The humeral component can include a ball-shaped head connected to a stem, and the glenoid component can include a concave surface against which the head articulates.

Total shoulder arthroplasty can be very traumatic to the patient. Not only must the shoulder be exposed and dislocated during surgery, but also the artificial prostheses implanted may not be well accepted by the human body. It is oftentimes difficult to provide long term fixation of artificial materials to natural bone. Accordingly, patients may face multiple operations and persistent pain and discomfort.

Both the materials and shapes of the artificial shoulder joints have varied, and have included the use of all metal, all polymer, metal-polymer hybrids, and metal coated polymer prostheses. These prostheses, however, failed to provide optimal results to patients. Proper bonding between the glenoid component and human bone has been particularly difficult to achieve.

Thus, there exists a need for a glenoid prosthesis having features that can better alleviate the possibility of loosening and prevent the need for revision surgery.

### SUMMARY OF THE INVENTION

The following presents a simplified summary of the invention in order to provide a basic understanding of some aspects of the invention. This summary is not an extensive overview of the invention. It is not intended to identify key or critical elements of the invention or to delineate the scope of the invention. Its sole purpose is to present some concepts of the invention in a simplified form as a prelude to the more detailed description that is presented later.

According to its major aspects and briefly stated, the present invention includes a glenoid prosthesis for use in total shoulder arthroplasty. The glenoid prosthesis has a concave articulating plate that can interact with the humeral component of an artificial shoulder joint, and a backing for connecting and bonding the prosthesis to the human scapula. The backing of the glenoid prosthesis can be made using hydroxyapatite, which forms a bond with human bone. The backing can further include means for connecting, such as pegs or a keel, to facilitate the fixation of the glenoid prosthesis with the scapula.

A feature of the present invention includes the use of a glenoid prosthesis having a hydroxyapatite backing. Hydroxyapatite is a complex phosphate of calcium, Ca₅(PO₄)₃OH, that occurs as a mineral and is the chief structural element of vertebrate bone. Consequently, this material is readily accepted by the human body and forms a natural and stable bond with human bone. Furthermore, hydroxyapatite promotes bone in-growth, which enhances the stability of the bond thereby permitting long term fixation of the glenoid prosthesis to the scapula.

Another feature of the present invention includes the use of a glendoid prosthesis having a hydroxyapatite backing including a connecting means, such as pegs or a keel. The use of pegs or a keel for mating with corresponding holes or a groove, respectively, that are formed within the scapula can further facilitate the proper orientation and bonding of the glenoid prosthesis to the scapula. Moreover, because the pegs or keel are also coated using hydroxyapatite, the bond formed between the pegs or keel and the bone is natural and stable.

Other features and their advantages will be apparent to those skilled in the art of shoulder arthroplasty from a careful reading of the Detailed Description of Preferred Embodiments, accompanied by the Figures.

### BRIEF DESCRIPTION OF THE DRAWINGS

In the drawings,

FIG. 1 illustrates a schematic view of the anatomy of a human shoulder with a glenoid prosthesis according to a first embodiment of the present invention;

FIG. 2 illustrates a perspective view of the glenoid prosthesis according to the first embodiment of the present invention;

FIG. 3 illustrates a side view of the glenoid prosthesis according to the first embodiment of the present invention;

FIG. 4 illustrates an exploded view of the glenoid prosthesis with the scapula according to the first embodiment of the present invention;

FIG. 5 illustrates a sectional view of the glenoid prosthesis connected to the scapula and showing the head of a humeral component of an artificial shoulder joint according to the first embodiment of the present invention;

FIG. 6 illustrates a perspective of the glenoid prosthesis according to a second embodiment of the present invention;

FIG. 7 illustrates a side view of the glenoid prosthesis according to the second embodiment of the present invention;

FIG. 8 illustrates an exploded view of the glenoid prosthesis with the scapula according to the second embodiment of the present invention; and

FIG. 9 illustrates a sectional view of the glenoid prosthesis connected to the scapula and showing the head of a humeral component of an artificial shoulder joint according to the second embodiment of the present invention.

### DETAILED DESCRIPTION OF PREFERRED EMBODIMENTS

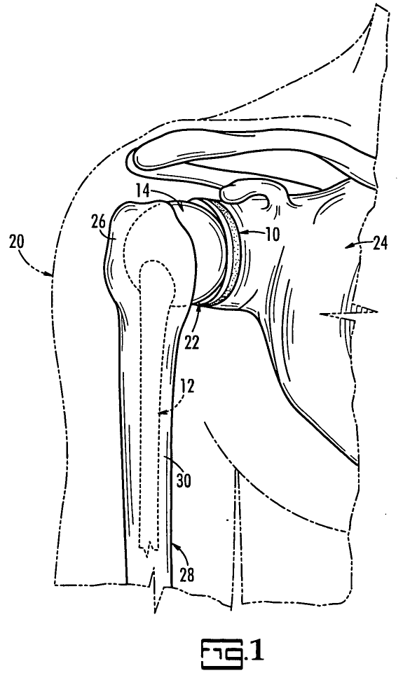

The present invention includes a glenoid prosthesis 10 for use in total shoulder arthroplasty. FIG. 1 illustrates the glenoid prosthesis 10 implanted in a human shoulder 20. In particular, the glenoid prosthesis 10 is positioned in a portion of the glenoid cavity 22 of the scapula 24 of the human body. As further shown, the glenoid prosthesis 10 is adjacent to and interacting with a humeral prosthesis, which can include a ball-shaped head 14 and a stem 12 that can be attached to the proximal end 26 of the humerus 28, and within the humerus shaft 30 of the body. The description and illustration of a humeral component is made merely for completeness and to place the glenoid prosthesis 10 of the present invention into context. Any number of various and standard humeral prostheses can be employed in combination with the glenoid prosthesis 10 of the present invention as long as the head 14 of the humeral prosthesis is generally sphere-shaped.

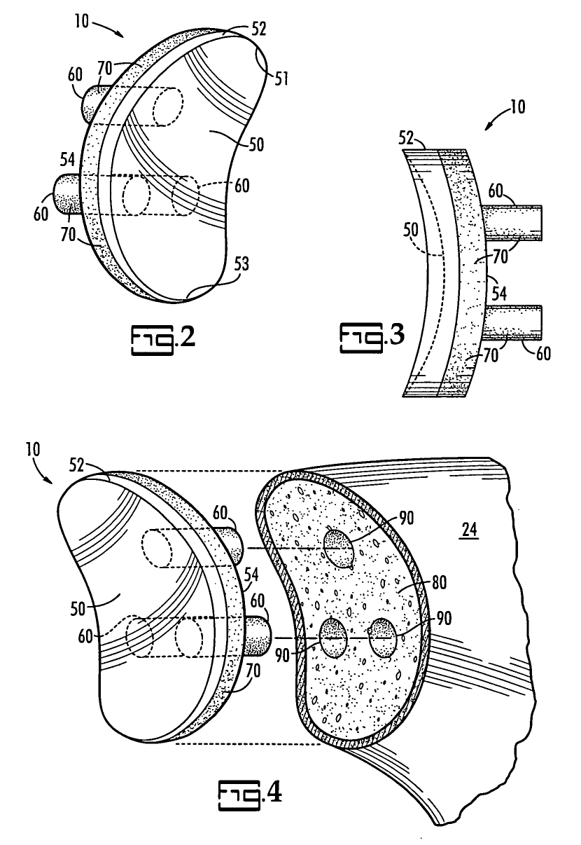

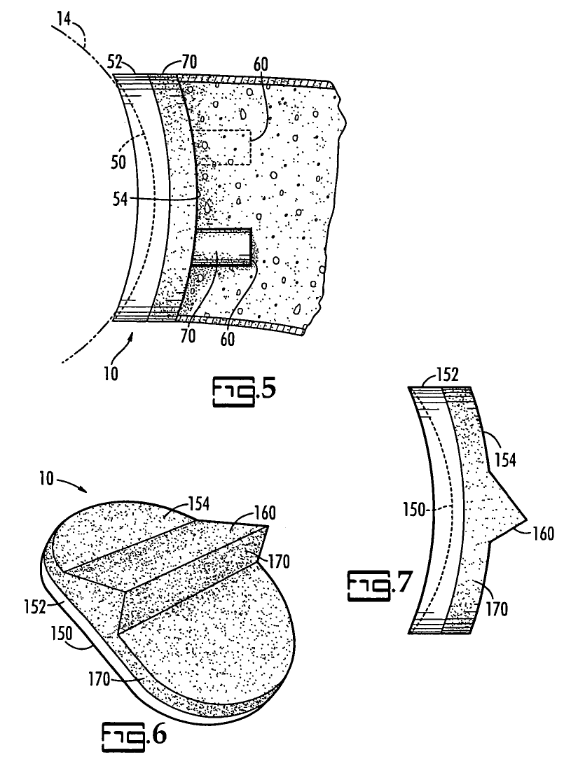

A first embodiment of the glenoid prosthesis 10 of the present invention is shown in further detail in FIGS. 2-5. The glenoid prosthesis 10 includes an articulating plate 52 having a concave surface 50 that faces the humerus 28 once the glenoid prosthesis 10 is implanted within a human body, and a backing 70 having a bonding surface 54 that faces the scapula 24 once the prosthesis 10 is implanted. Preferably, the articulating plate 52 of the present invention is made of ultra-high molecular weight, highly cross-linked polyethylene. The backing 70 is preferably made of hydroxyapatite. Although various methods can be employed to form the backing 70, one method includes spraying hydroxyapatite under pressure onto the articulating plate 52.

As discussed, a feature of the present invention includes the use of the hydroxyapatite backing 70. Hydroxyapatite is a complex phosphate of calcium, Ca₅(PO₄)₃OH, that occurs as a mineral and is the chief structural element of vertebrate bone. Consequently, this material is readily accepted by the human body and forms a natural and stable bond with human bone. Furthermore, hydroxyapatite promotes bone in-growth, which enhances the stability of the bond thereby permitting long term fixation of the glenoid prosthesis to the scapula.

As shown in FIG. 2, the shape of the articulating plate 52 can generally be egg-shaped including a narrower superior portion 51 and a broader inferior portion 53 in relation to the superior and inferior portions of the glenoid cavity 22 when the glenoid prosthesis 10 is implanted. This shape resembles the natural shape of the glenoid cavity 22, so as to facilitate the proper mating and connection between the glenoid prosthesis 10 and the scapula 24. Although dimensions may vary according to the size of a particular patient's glenoid cavity 22, the superior portion 51 can have a width of about 1.5 cm to about 2 cm, and the inferior portion 53 can have a width of about 2.5 cm. Further, the articulating plate 52 can have a total length as measured from the superior portion 51 to the inferior portion 53 of about 3 cm.

Optionally, the backing 70 can include a plurality of pegs 60 that extend from the bonding surface 54. The plurality of pegs 60 can include three pegs that are positioned approximately in a triangle formation with one peg located above and between two laterally spaced pegs. Further, the pegs can be located approximately in the medial portion of the backer 70 and can extend out approximately perpendicular to the plane of the backer 70. If the plurality of pegs 60 is included, these pegs are also preferably made of or coated with hydroxyapatite. Accordingly, the pegs can be formed integrally with the backer 70.

Although surgical procedures employed in total shoulder arthroplasty, the glenoid prosthesis 10 of the present invention can be implanted by generally exposing and dislocating the patient's shoulder joint. Thereafter, the glenoid prosthesis 10 is fixed to the scapula 24. In particular, the subchondral bone of the scapula 24 is resected in an amount substantially equal to the height of the glenoid prosthesis 10 and a resected surface 80 of the scapula 24 is provided. If the glenoid prosthesis 10 includes the plurality of pegs 60, the surgeon can thereafter drill a plurality of peg holes 90 that are positioned and dimensioned to receive and mate with the plurality of pegs 60. When properly fixed, an implanted humeral prosthesis can be placed adjacent to the glenoid prosthesis 10 so that the humeral head 14 can articulate with the concave surface 50 of the glenoid prosthesis 10.

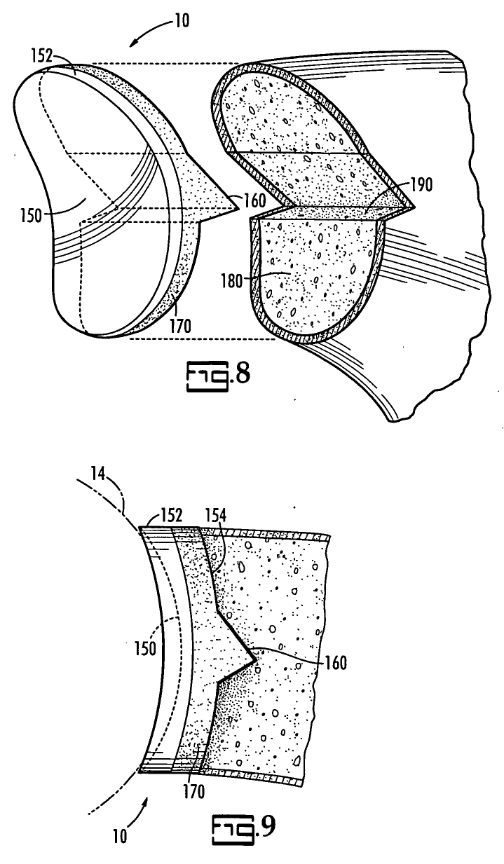

FIGS. 6-9 illustrate a second embodiment of the present invention. As shown, The glenoid prosthesis 10 includes an articulating plate 152 having a concave surface 150 that faces the humerus 28 once the glenoid prosthesis 10 is implanted within a human body, and a backing 170 having a bonding surface 154 that faces the scapula 24 once the prosthesis 10 is implanted. Preferably, the articulating plate 152 of the present invention is made of ultra-high molecular weight, highly cross-linked polyethylene. The backing 170 is preferably made of hydroxyapatite. Although various methods can be employed to form the backing 170, one method includes spraying hydroxyapatite under pressure onto the articulating plate 152.

Optionally, the backing 170 can include a keel 160. In relation to the glenoid prosthesis 10 when it is implanted, the keel 160 can extend horizontally between opposing side edges of the backer 170 and from the bonding surface 154. The keel 160 can be located approximately in the medial portion of the backer 170. Alternatively, the keel 160 can also be contained within the opposing side edges of the backer 170, and can comprise about the inner third of the total width of the glenoid prosthesis 10. This embodiment will resemble a shark fin. If the keel 160 is included, it is preferably made of or coated with hydroxyapatite. Accordingly, the keel 160 can be formed integrally with the backer 170.

As previously discussed, a feature of the present invention includes the use of the hydroxyapatite backing including a connecting means, such as the plurality of pegs 60 or the keel 160. The use of pegs or a keel for mating with corresponding holes or a groove, respectively, that are formed within the scapula 24 can further facilitate the proper orientation and bonding of the glenoid prosthesis 10 to the scapula 24. Moreover, because the pegs or keel are also made using hydroxyapatite, the bond formed between the pegs or keel and the bone is natural and stable.

The glenoid prosthesis 10 of the second embodiment can be implanted similarly to the procedure described above. First, the patient's shoulder joint is generally exposed and dislocated. Thereafter, the glenoid prosthesis 10 is fixed to the scapula 24. In particular, the subchondral bone of the scapula 24 is resected in an amount substantially equal to the height of the glenoid prosthesis 10 and a resected surface 180 of the scapula 24 is provided. If the glenoid prosthesis 10 includes the keel 160, the surgeon can further resect a groove 90 that is positioned and dimensioned to receive and mate with the keel 160. When properly fixed, an implanted humeral prosthesis can be placed adjacent to the glenoid prosthesis 10 so that the humeral head 14 can articulate with the concave surface 150 of the glenoid prosthesis 10.

It will be apparent to those skilled in the art that many changes and substitutions can be made to the preferred embodiments herein described without departing from the spirit and scope of the present invention.

What is claimed is:

1. A glenoid prosthesis, comprising:
an articulating plate having a concave surface, wherein said concave surface is shaped to articulate with the head of a humerus of a shoulder joint; and
a backer attached to said articulating plate and having a bonding surface for engaging a surface of the subchondral bone of a scapula, wherein said backer is made of hydroxyapatite.

2. The glenoid prosthesis as recited in claim 1, where said backer includes a plurality of pegs.

3. The glenoid prosthesis as recited in claim 2, wherein said plurality of pegs is three pegs.

4. The glenoid prosthesis as recited in claim 3, wherein said three pegs are positioned in a triangle formation.

5. The glenoid prosthesis as recited in claim 3, wherein said three pegs are located within the medial portion of said backer.

6. The glenoid prosthesis as recited in claim 2, wherein said plurality of pegs is made of hydroxyapatite.

7. The glenoid prosthesis as recited in claim 2, wherein said plurality of pegs is coated with hyroxyapatite.

8. The glenoid prosthesis as recited in claim 1, wherein said backer includes a keel.

9. The glenoid prosthesis as recited in claim 8, wherein said keel extends horizontally between opposing side edges of said backer.

10. The glenoid prosthesis as recited in claim 8, wherein said keel is made of hydroxyapatite.

11. The glenoid prosthesis as recited in claim 8, wherein said keel is coated with hyrdoxyapatite.

12. The glenoid prosthesis as recited in claim 1, wherein said articulating plate is made of ultra-high molecular weight, highly cross-linked polyethylene.

13. The glenoid prosthesis as recited in claim 1, wherein said articulating plate includes a superior portion and an inferior portion, and wherein said inferior portion is broader than said superior portion.

14. A method for making a glenoid prosthesis, comprising;
providing an articulating plate having concave surface;
providing an amount of hydroxyapatite;
spraying said hydroxyapatite onto said articulating plate under pressure to form a backer; and
forming a plurality of pegs on said backer.

15. The method as recited in claim 14, wherein said articulating plate is made of ultra-high molecular weight, highly cross-linked polyethylene.

16. The method as recited in claim 15, wherein said plurality of pegs is three pegs.

17. The method as recited in claim 16, wherein said three pegs are positioned in a triangle formation.

18. The method as recited in claim 14, wherein said three pegs are coated with said hydroxyapatite.

19. A method for making a glenoid prosthesis, comprising;
providing an articulating plate having concave surface;
providing an amount of hydroxyapatite;
spraying said hydroxyapatite onto said articulating plate under pressure to form a backer; and
forming a keel on said backer.

20. The method as recited in claim 19, wherein said keel extends horizontally between opposing side edges of said backer.
